SEQUENCE LISTING (5 sequences)

SEQ ID NO: 1 - DNA, 608 bp, Curvularia lunata
  misc_feature: NCBI GENBANK ACCESSION AY034827
ggcacgaggcacgcttgaacaactaccgtctataccacctattcaacctacactcaccttatctcatcacaatgggtttcgaacagggcgatgctaagaagggtgccaacctcttcaagacccgctgcgctcagtgccacaccctgaaggccggcgagggcaacaagattggccctgagctccacggtctctttggccgcaagactggttccgtcgctggctactcatacaccgacgccaacaagcagaagggtatcgagtggaaccatgacactctgttcgagtacctcgagaaccccaagaagtacattcccggcaccaagatggcttttggcggtctcaagaagcccaaggaccgcaacgacctcatcaccttccttgagcaggagaccaaataagcgtctctgtgcccccgttgacgttacctttctccgacgcgccatgacccttctctctaataccttggaccaaaatcttctttggattgtaccaacatgaatgatagattcaaaagatggaggccgtgggcgcaacccggagcgcatttgctgtttcatgtagaatatgttttggggtatcattttgcgggccgttttcgatcaggtgca

SEQ ID NO: 2 - DNA, 327 bp, Curvularia lunata
  misc_feature: NCBI GENBANK ACCESSION  AY034827 REGION 72..398
atgggtttcgaacagggcgatgctaagaagggtgccaacctcttcaagacccgctgcgctcagtgccacaccctgaaggccggcgagggcaacaagattggccctgagctccacggtctctttggccgcaagactggttccgtcgctggctactcatacaccgacgccaacaagcagaagggtatcgagtggaaccatgacactctgttcgagtacctcgagaaccccaagaagtacattcccggcaccaagatggcttttggcggtctcaagaagcccaaggaccgcaacgacctcatcaccttccttgagcaggagaccaaataa

SEQ ID NO: 3 - protein, 108 aa, Curvularia lunata
  MISC_FEATURE: NCBI ACCESSION   AAK67492
MGFEQGDAKKGANLFKTRCAQCHTLKAGEGNKIGPELHGLFGRKTGSVAGYSYTDANKQKGIEWNHDTLFEYLENPKKYIPGTKMAFGGLKKPKDRNDLITFLEQETK

SEQ ID NO: 4 - DNA, 33 bp, Artificial Sequence
  Description of Artificial Sequence Synthetic primer derived from Curvularia lunata
gatccgaattctatgggtttcgaacagggcgat

SEQ ID NO: 5 - DNA, 30 bp, Artificial Sequence
  Description of Artificial Sequence Synthetic primer derived from Curvularia lunata
aagcttaagctttttggtctcgtgctcctg

(19) United States
(12) Patent Application Publication
(10) Pub. No.: US 2007/0224608 A1
(43) Pub. Date: Sep. 27, 2007

(54) Recombinant 12-KDA protein useful for the detection of respiratory allergies

(75) Inventors: Naveen Arora, Delhi (IN); Bhanu Pratap Singh, Delhi (IN); Vidhu Sharma, Delhi (IN)

Correspondence Address: BIRCH STEWART KOLASCH & BIRCH, PO BOX 747, FALLS CHURCH, VA, 22040-0747, US

(73) Assignee: COUNCIL OF SCIENTIFIC AND INDUSTRIAL RESEARCH, New Delhi (IN)

(21) Appl. No.: 11/585,924

(22) Filed: Oct. 25, 2006

(30) Foreign Application Priority Data
Oct. 25, 2005 (IN) 1537/DEL/2005

(51) Int. Cl. C40B 40/08; C40B 40/10; C12Q 1/68; G01N 33/569; C07H 21/04; C07K 14/37

(52) U.S. Cl. 435/6; 435/7.1; 435/69.3; 435/254.1; 530/371; 536/23.7; 435/7.31

(57) ABSTRACT

The present invention discloses the detection of an important 12K-Da protein having cross-reactivity amongst different prevalent allergenic grasses and fungi can be useful for detection of respiratory allergies. Conventionally, the whole extracts that are used for diagnosis are unable to specifically detect the causative agents. In addition, they are also responsible for additional non-specific sensitivities in patients to other components present in the extract. If a single cross-reactive protein is available, it can replace large number of extracts used for detection of raised IgE levels in allergy by ELISA, immunoblotting and the likes. Further, number of pricks would be reduced and this would benefit both patient and clinicians. It is further realized that production of such a protein by recombinant methods can lead to its availability in pure form and bulk amounts required for routine diagnosis.

### FIELD OF THE INVENTION

The present invention relates to a recombinant 12 kDa protein useful for the detection of respiratory allergies. The invention particularly relates to detection of the respiratory allergies caused by fungal spores and grass pollen using the said protein.

### BACKGROUND OF THE INVENTION

The term “allergy” coined by Von Pirquet (1906) is defined as altered immunologic reactivity to foreign particles. The foreign agents causing altered immunologic reactivity are called allergens, which includes a broad spectrum of substances e.g. proteins, glycoprotein, lipoproteins etc derived from diverse sources such as pollens, fungal spores, insects, dust mites, animal danders, foods, etc. Pollen grains and fungal spores are the main constituents of the aerospora. They are significant cause of allergic diseases afflicting more than 25% of the atopic subjects. These foreign substances can trigger the release of mediators from immune system leading to inflammatory and other allergic reactions.

Allergy is detected clinically by skin testing and ELISA. The fungal extracts generally used for skin testing are complex mixture of proteins, carbohydrates, pigments, toxins, etc. They contain both relevant and non-relevant components that might sensitize the patient and eventually evoke anaphylaxis. Another factor that adds complexities to diagnosis of fungal allergy, is cross-reactivity among allergens from different sources. Cross reactivity is due to the presence of similar protein components and/or epitopes shared by different fungal species. Studies on cross reactivity have shown antigenic/allergenic relationship among species of fungi such as Curvularia, Cladosporium, Fusarium, Pencillium and Aspergillus [1]. Curvularia lunata has been shown to be an important allergy causing fungi also responsible for life threatening Allergic bronchopulmonary aspergillosis (ABPA) like symptoms in many patients [2].

Due to complexities involved in standardizing the large number of extracts, use of recombinant allergens have now begun for diagnostic purposes, e.g. use of rAsp f 1, rAsp f 3 and rAsp f 6 has made it possible to diagnose differentially between ABPA and A. fumigatus sensitized asthmatics, (Hemmann et al). For three reasons, diagnosis with recombinant allergen is advantageous over heterogeneous crude allergen extract [3,4]. First, it provides pure, standardized and consistent allergen preparations. Secondly, skin tests with these preparations are free of false positive or false negative i.e. non-specific reactions. Thirdly, it allows substantial evidence of patient's specific reactivity against a particular allergen and thus helps in better understanding of the disease causing components. Isolation and purification of recombinant cross-reactive allergens would lead to a better and easier way of diagnosis. Since, using cross-reactive allergens would reduce the number of extracts used for skin testing [5]. The recombinant form of these cross-reactive allergens would improve sensitivity of such diagnosis [6]. An important criterion for such applications is that there should be equivalent immuno-biochemical properties of the recombinant allergen and its native counterpart. Many recent reports have suggested the same e.g. Grendelmeier P S et al have reported [7] that Art v 1 restores its properties after making it recombinant. Similarly, a recent report [8] from Jeong KY group have shown the similar properties of recombinant and native Bla g 7, an allergen from German cockroach.

### OBJECTS OF INVENTION

The main object of the invention is thus to provide a recombinant 12 K-Da protein useful for the detection of respiratory allergies.

Another object of the present invention is to provide a method for detection of respiratory allergies using the said recombinant 12 kDa protein.

Still another object of the invention is to provide novel primers for sequencing and expression of the disclosed 12 kDa protein by recombinant methods.

Yet another object of the invention is the expression and purification of recombinant 12 kDa protein.

### SUMMARY OF THE INVENTION

The invention discloses the detection of respiratory allergies using a recombinant 12-kDa protein. The present invention is based on the fact that there is a need of a single cross-reactive protein capable of replacing large number of extracts used for detection of raised IgE levels in allergy by ELISA, immunoblotting and the likes. It is further based on the realization that such a cross-reactive protein will reduce the number of pricks, a patient gets during allergy skin testing, thus providing a single representative of large number of allergen extracts used. It is further realized that production of such a protein by recombinant methods can lead to its availability in pure form and bulk amounts required for routine diagnosis. In extension to the fact mentioned above, the resemblance of such a recombinant protein to its native form is an additional benefit forming the basis of its use clinically.

Accordingly, the present invention provides a recombinant 12 kDa fungal protein useful for detection of respiratory allergies, the said protein exhibiting the following characteristics:
a) a protein having mRNA sequence of SEQ ID 1 (NCBI ACCESSION NO. AY034827) and coding sequence (CDS) of SEQ ID 2 (NCBI ACCESSION NO. AY034827),
b) the translated protein sequence having SEQ ID 3 (NCBI ACCESSION NO. AAK67492),
c) resolves on SDS-PAGE as a protein with molecular weight 12 kDa,
d) having an iso-electric point of 9.5 as determined by isoelectric focusing,
e) having UV-visible absorbance peaks at 411 nm and 511 nm,
f) with CD spectra having characteristic double minima in the range of 210 nm-220 nm signifying high alpha helical content,
g) with melting temperature in the range of 57-58° C. as found by CD spectra,
h) is recognized by commercially available and raised specific polyclonal antibodies,
i) is having allergenic reactivity in patient's sera and which is three to four times that of healthy controls, as confirmed by ELISA and immunoblot,
j) is cross-reactive among grasses and fungi as confirmed by ELISA, immunoblot and ELISA inhibition,
k) is having comparable activity with its native form purified from fungus as confirmed by SDS-PAGE, immunoblot, ELISA, ELISA inhibition, absorbance and CD spectra,

The disclosed recombinant 12-kDa protein is highly cross-reactive in grasses and fungi as tested by ELISA inhibition. EC₅₀ required for 50% loss of IgE binding activity is in the range of 1-1.5 ng.

In an embodiment of invention, the cDNA library of fungus was constructed in commercially available λZAP vector and the like.

In still another embodiment, the fungus for cDNA library was selected from Curvularia lunata [MTCC 2030], Alternaria alternate [MTCC 1362], Epicoccum nigrum [MTCC 2129] and Fusarium solani [MTCC 1756].

In yet another embodiment of the invention, the screening of cDNA library for locating the protein of interest was carried out with pooled sera of patients allergic to Curvularia lunata and the like.

In still another embodiment of the invention, the mRNA sequence SEQ ID 1 (NCBI ACCESSION NO. AY034827) and its coding sequence (CDS) SEQ ID 2 (NCBI ACCESSION NO. AY034827) were obtained using known primers.

In still another embodiment of the invention, the protein sequence obtained by translating the coding sequence SEQ ID 3 (NCBI ACCESSION NO. AAK67492) was computationally compared with known sequences available in databank using ClustalW and BLAST and the like.

In yet another embodiment of the invention, novel primers of SEQ ID NOS. 4 and 5 were designed for sub-cloning the SEQ ID NO. 2.

In still another embodiment of the invention, the protein of SEQ ID 3, was expressed in E.coli prokaryotic expression vector and the like.

In still another embodiment of the invention, the purification of the recombinant protein was carried out using two steps comprising metal affinity chromatography and Gel exclusion chromatography and the like.

In yet another embodiment of the invention, the said protein resolved as 12 kDa protein on SDS-PAGE, was recognized by commercial and raised antibodies.

In still another embodiment of the invention, the allergenic properties of the recombinant protein were assessed by ELISA, immunoblot, ELISA inhibition and the like.

In still another embodiment of the invention, the native form of the disclosed allergen was purified using two-step method comprising cation exchange chromatography using CM cellulose and the like and gel exclusion chromatography using Sephadex G50 and the like.

In still another embodiment of the invention, the disclosed recombinant allergen was compared to its native counterpart by physiochemical viz. CD and absorption spectra and like and immunological methods viz. immunoblot, ELISA, ELISA inhibition and the like.

In still another embodiment of the invention, the cross-reactivity of the disclosed recombinant allergen was compared with fungi viz. A. alternata, E. purpurascens, F. solani, C. albicans and the like by ELISA, Immunoblot, ELISA inhibition and the like.

In still another embodiment of the invention, the cross-reactivity of the disclosed recombinant allergen was checked with grass pollen viz. Lolium perenne, Poa pretense, Phleum pretense, Imperata cylindrica Pennisetum sp., Rye grass, Zea Mays and Cenchrus and the like by immunoblot, ELISA and ELISA inhibition using pooled and individual allergic sera as well as commercial and raised antibodies against disclosed protein.

### BRIEF DESCRIPTION OF THE FIGURES

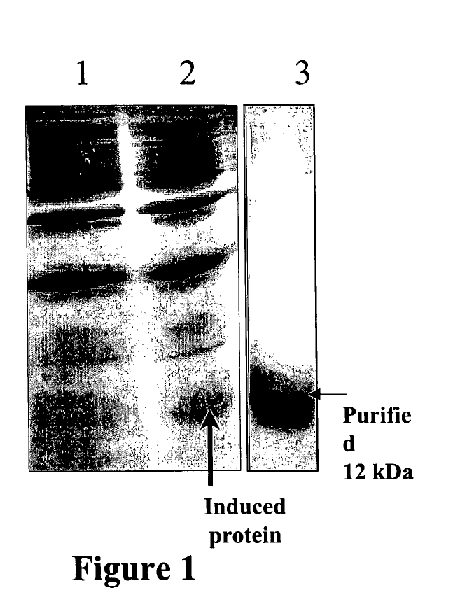

1. FIG. 1 depicts the 12% SDS-PAGE profile of uninduced (Lane 1) and induced (Lane 2) recombinant protein. Lane 3 shows the profile of purified 12 kDa.

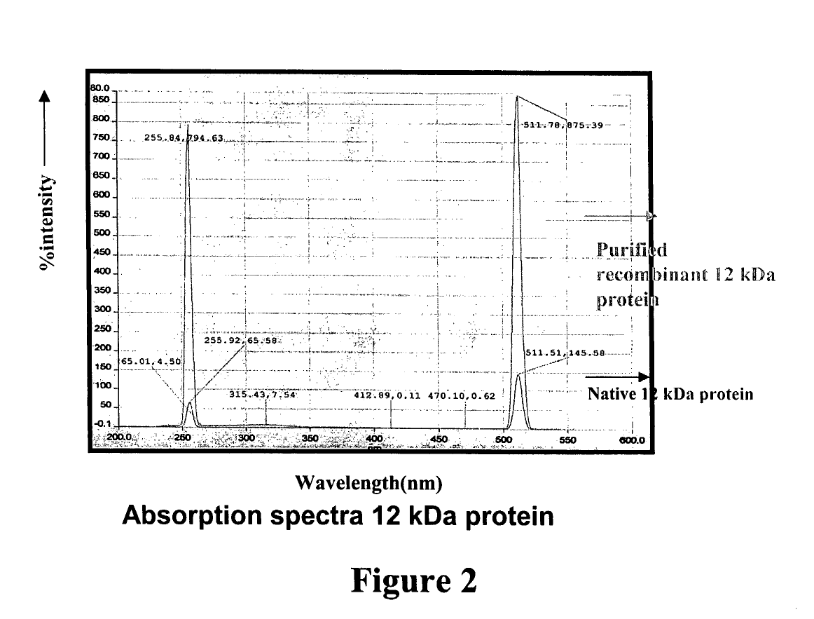

2. FIG. 2 shows the absorption spectra of purified 12-kDa protein. The prominent absorption peaks were seen at 410 nm, 470 nm and 511 nm.

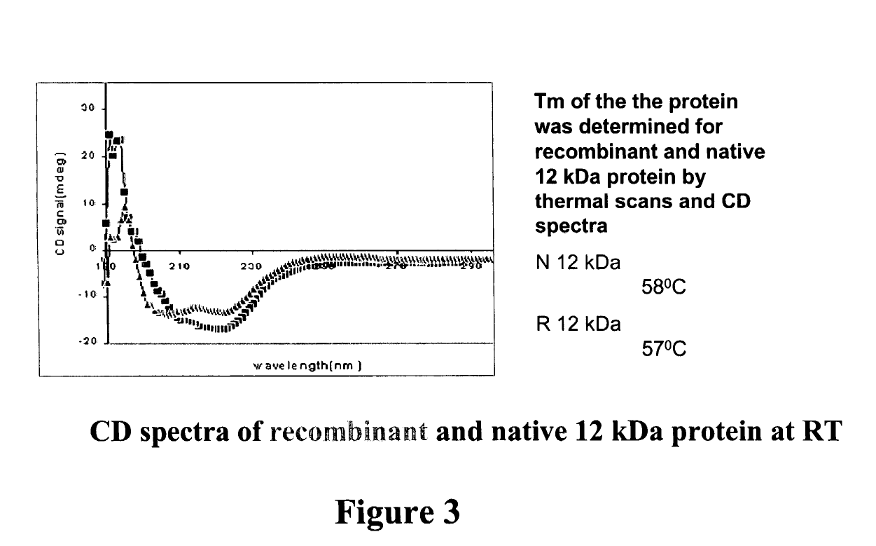

3. FIG. 3 depicts the CD spectra of both recombinant and native form of 12-kDa protein. The equivalent minima and maxima were observed indicating comparable secondary structure.

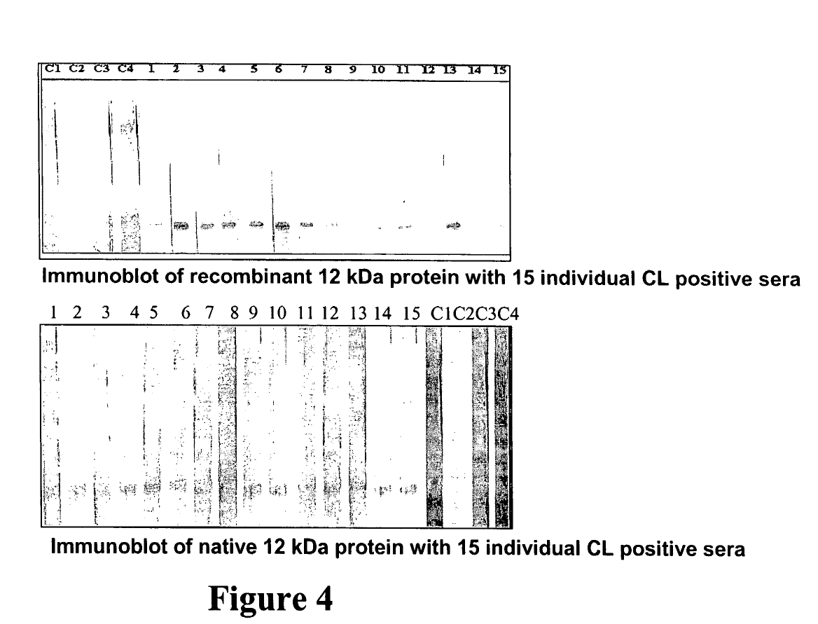

4. FIG. 4 exhibits the immunoblot of both recombinant and native form of 12-kDa allergen with 15 individual patient sera and 4 control healthy sera.

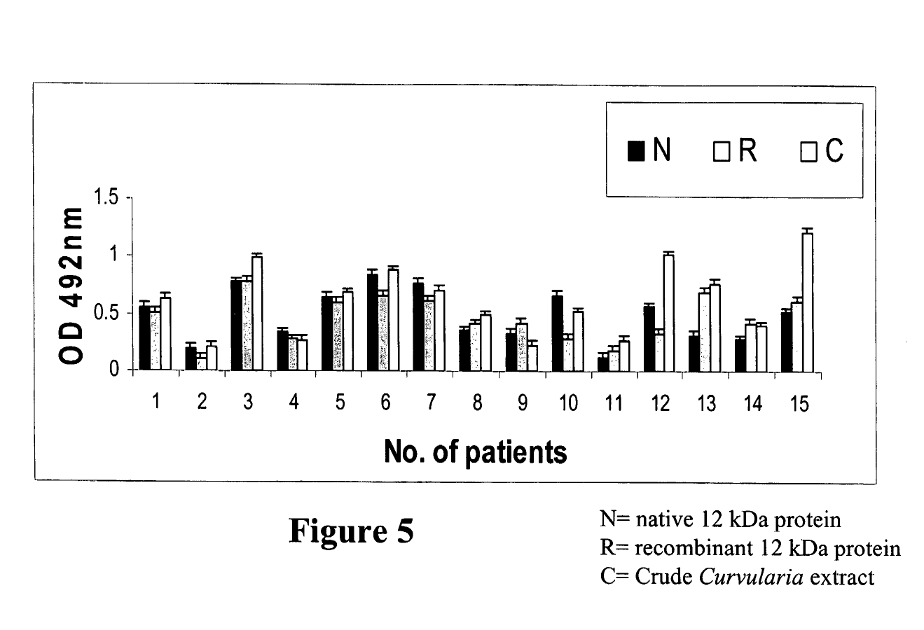

5. FIG. 5 shows the IgE specific ELISA with crude extract of Curvularia and both recombinant and native form of 12-kDa protein.

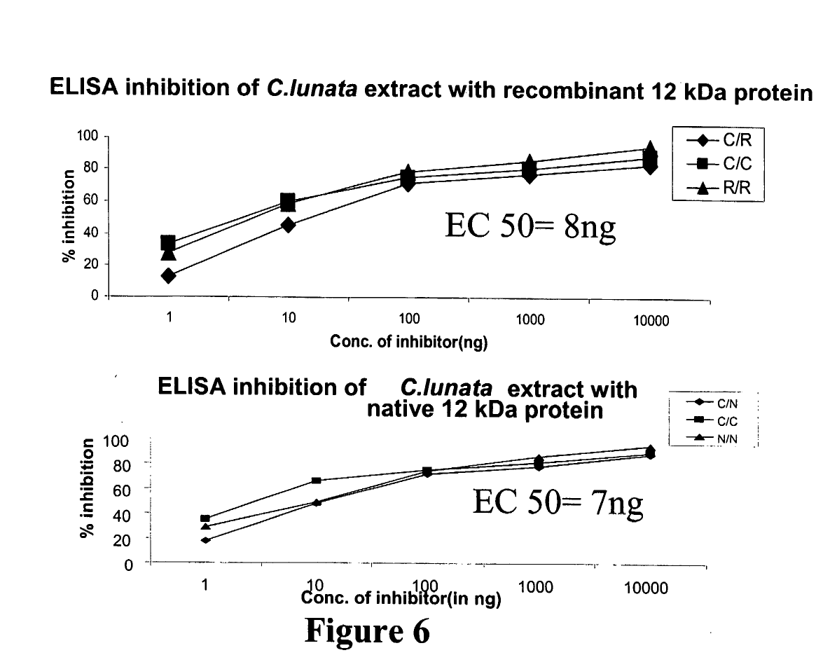

6. FIG. 6 depicts the ELISA inhibition of both recombinant and native form of 12-kDa proteins with crude Curvularia extract. Both the forms of 12-kDa protein required comparable amount (i.e. 8 ng for recombinant and 7 ng for native) for 50% inhibition. This indicates the comparable nature of both these forms.

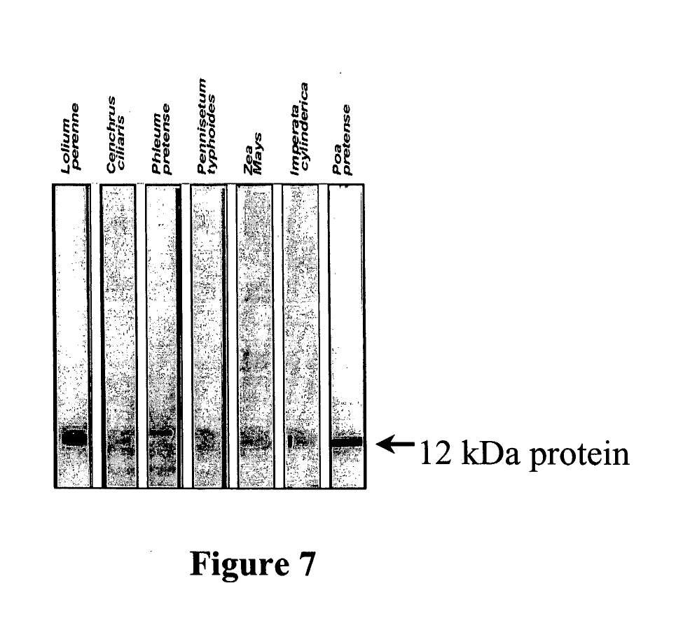

7. FIG. 7 exhibits the cross-reactivity of 12-kDa protein with various prevalent Indian and Western grass positive patient sera.

### BRIEF DESCRIPTION OF THE TABLES

Table 1: ELISA values carried out with 12-kDa allergen with patient sera of fungal positive sera.
Table 2: ELISA values, carried out with fungal negative and grass positive sera. These values show the cross-reactive nature of 12-kDa protein

### DETAILED DESCRIPTION OF THE INVENTION

1. Total RNA isolation: C.lunata grown in Sabouraud's broth for 4 days was used for isolating total RNA. Fresh CL culture was separated from the medium and washed with diethyl pyrocarbonate (DEPC) treated water. The total RNA was isolated by mono-phasic solution of phenol and guanidium isothiocyanate using Trizol reagent (Life Technologies). The yield was determined spectrophotometrically and the purity was determined on formaldehyde agarose RNA gel. Seven hundred twenty microgram of RNA was obtained from 3 g of spore mycelial mass (24 μg/100 mg spore mycelial mass).
2. mRNA isolation: Messenger RNA was isolated from total RNA using oligo (dT) cellulose column commercially available. From total RNA, 7 μg mRNA was obtained and cDNA library was constructed using 5 μg mRNA.
3. Construction of cDNA library: A cDNA expression library was constructed in Uni ZAP XR vector commercially available with poly (A)RNA. The cDNA molecules were synthesized by using 50 base oligonucleotide primer and reverse transcriptase. Using commercial primers provided with kit. The cDNA obtained was ligated with Eco R I adapters. The cDNA was digested with Xho I, fractionated and 0.5-2.0 kb fragments were ligated into Uni-ZAP XR vector. The ligation mix was packaged using commercial Gigapack III Gold packaging extract at 22° C. for 2 h. The percentage of non-recombinant background plaques was determined on NZY plates containing IPTG and X-gal. The background of non-recombinant phages was 0.6%. The Escherichia coli strain XL-1 Blue was used as host for amplification and screening of the library. The titer of the library was 1×10⁹.
4. Escherichia coli strain: E.coli XL-1-Blue MRF′ strain is RecA− and contained F′ episome, essential for Uni-ZAP XR vector. The F′ episome present in E.coli XL-1-Blue MRF′ strain is required for phage infection and contains (a) enzymatically inactive β-galactosidase gene required for enzyme based non-recombinant selection strategy (b) expresses the genes forming F′ pili found on the surface of the bacteria (c) contains lac repressor gene.
5. E.coli SOLR strain was used for plating excised phagemids. Ex ASIST™ interference resistance helper phage was used for excision of the pBluescript phagemids from the Uni-ZAP XR vector.
6. Patient's sera: The serum from 10-nasobronchial allergy patients hypersensitive to Curvularia as determined by intradermal tests were collected pooled and used for screening the cDNA library.
7. Screening of cDNA library with patient's sera: The cDNA library was screened by using a pre-absorbed human serum pool with E. coli XL-1 Blue lysate. The phages were used to infect E. coli XL-1 Blue cells and plated on 90 mm NZY agar plate (density 300-400 plaques/plate). Screening was performed using pre-absorbed patient's serum.
8. PCR Amplification and DNA Sequencing: The positive clones were isolated and the phage stock was used to infect E. coli XL-1 Blue cells. The plaques obtained were transferred onto the nitrocellulose filter, presoaked with IPTG. Rescreening was performed using pre-absorbed patient's serum. The cDNA insert in the phage was amplified by polymerase chain reaction with T3/T7 primers. The amplified product was purified using commercial DNA isolation kit. An overnight culture of E. coli XL-1 Blue cells was co-infected with the cloned phage and helper phage to convert the phage into the phagemid. The plasmid DNA was isolated and was used as a template for DNA sequencing using T3/T7 universal commercial primers and sequences submitted to NCBI GENBANK (SEQ ID # 1 and 2).
9. Computational analysis: ORF of one of the positive clone was obtained using DNASTAR program, translated and homology search was done using NCBI-BLAST both at nucleotide and protein level. ORF of the sequence was 327 bp encoding 108 amino acids (SEQ ID # 3) Molecular mass determined computationally was 12 kDa and pl was 9.5.
10. Expression and purification of recombinant form of 12-kDa protein in E.coli: The ORF obtained was amplified using primer SEQ ID # 4 and 5. The PCR product was digested with restriction enzymes, ligated into pET22b+ vector. The plasmid containing the insert was transformed into BL-21 E.coli cells. The his-tagged recombinant 12 kDa protein was purified using Ni-NTA affinity purification with a yield of about 0.5 mg/l of culture and further by gel filtration chromatography. This protein resolved as 12-kDa protein on SDS-PAGE.
11. Purification of native form of 12-kDa protein: The 13-day old C. lunata fungal mat grown in Sabraoud's media was harvested, lyophilized and stored at −20° C. for purification of native form of this 12-kDa protein. The antigens/allergens from Curvularia were extracted 1:20 (w/v) in 50 mM ammonium bicarbonate (NH₄HCO₃) pH-8. The protein concentration was estimated using Lowry's method. Lyophilized protein was dissolved in sodium phosphate buffer and kept for binding with swollen and equilibrated CM-C50 cation exchange resin (Pharmacia) at 4° C. overnight. The elution of protein was carried out using increasing ionic strength of NaCl gradient in phosphate buffer. The protein was eluted using 0.5M NaCl in 20 mM sodium phosphate buffer. The fractions containing 12 kDa protein were loaded on equilibrated Sephadex G-50 column. The activity of the native protein was estimated by both absorption spectra and immunoblot with raised antibody.
12. Comparative Study of recombinant and native forms of 12 kDa:
   A. Immunoblot of 12 kDa protein with allergic patient sera:
   The purified 12 kDa protein were resolved by SDS-PAGE and transferred onto nitrocellulose. The membranes were washed and incubated overnight at 4° C. with 15 individual hypersensitive patient sera. After washing the strips with PBS-Tween20, anti-human IgE peroxidase or protein G peroxidase or anti-rabbit IgG peroxidase were added to the respective blots. After incubation at 37° C., membranes were washed and developed with Diaminobenzidine in acetate buffer. All the patients' sera reacted positively to both the proteins indicating that they are major allergens.
   B. ELISA of purified 12 kDa protein with allergic patient sera
   IgE specific ELISA was performed with both the forms of 12-kDa protein (i.e. recombinant and native) using known methods [12] to determine its allergenic potency. Purified protein was coated on microtiter plate in carbonate buffer (pH9.6) and was kept overnight at 4° C. The plate washed with PBS-Tween20, blocked and incubated overnight with 120 different fungal allergic patient sera. Appropriate healthy individual sera were also used as controls. The plates were washed with and incubated for 3 hrs at 37° C. with anti-human IgE peroxidase conjugated. The color was developed with o-phenylene diamine in sodium citrate buffer. The reaction was stopped after 30 minutes with 50 μl of 5N H₂SO₄ and absorbance was read at 490 nm in ELISA reader. IgE ELISA value was comparable for the proteins and ranged from 0.6-1.2
   C. ELISA inhibition of 12 kDa recombinant protein with crude Curvularia extract
   For ELISA inhibition study, crude extract and recombinant protein was coated on ELISA plates separately. The plates were washed, blocked and incubated overnight at 4° C. with pre-incubated mixture of sera having different concentration of recombinant protein. The plates were washed and incubated with anti-human IgE peroxidase labeled. The color was developed with OPD and the reaction was stopped with 5N H₂SO₄ and read at 490 nm in ELISA reader. Around 5 ng of recombinant 12 kDa protein was required to obtain 50% inhibition. ELISA inhibition study indicates that they are having the same allergenic potential.
   D. Absorption spectra of purified recombinant protein
   The absorption spectrum of 12 kDa protein was to find the characteristic absorption maxima peaks of heme containing proteins and shows the peaks at 410 nm, 510 nm and 550 nm.
13. Circular dichroism of 12-kDa protein: CD spectra were carried out with 1 mg/ml of each recombinant and native protein in 20 mM phosphate buffer in the far-UV range. Thermal scans in the range of 10-100° C. were also carried out to find the melting temperature (Tm) of recombinant 12 kDa protein.
14. Detection of 12-kDa protein in fungi: Specific IgE ELISA was performed with fungal hypersensitive sera. Purified protein was coated on the plates and incubated overnight at 4° C. in carbonate buffer (pH9.6). The plate washed with PBS containing 0.05% Tween20, blocked and incubated overnight with each of 118 different fungal allergic patient's sera. Healthy sera from 20 individuals were used as negative control. The plates were washed, incubated for 3 hrs at 37° C. with anti-human IgE peroxidase. After washing, color was developed with o-phenylene diamine and absorbance was read at 490 nm in ELISA reader. The specific IgE values for 12-kDa recombinant protein with 118 fungal allergy patients The specific IgE values for 12 kDa protein for most of the patients' sera ranged from 0.7-1.6 compared to 20 healthy controls i.e. 0.2-0.3. It was found that patients positive to A.alternata, E.nigrum, F.solani and A.fumigatus showed slightly higher specific IgE values as compared to other fungi. This data highlights the cross-reactive nature of this protein among fungi.
15. 12 kDa protein as a cross-reactive allergen in tropical and temperate grasses:

To establish the presence of r-12 kDa protein in different grasses, ELISA and immunoblot of various Indian and Western grasses was done. Grass extracts used were Lolium perenne, Poa pretense, Phleum pretense, Imperata cylindrica, Pennisetum sp., Rye grass, Zea Mays and Cenchrus. Protein extracts were coated on microtiter plate and ELISA was done as earlier, using antibodies raised in mouse. Anti-mouse IgG peroxidase labeled as secondary antibody. These extracts were separated on SDS-PAGE and transferred on nitrocellulose membrane and immunoblot performed as earlier using antibody raised in mouse against r 12 kDa protein. It showed the presence of r-12 kDa protein in grass extracts viz. Lolium perenne (Lol p), Poa pretense (Poa p), Phleum pretense (Phl p), Imperata cylindrica (Imp c), Pennisetum sp., Zea Mays and Cenchrus. Further, allergenicity of recombinant 12 kDa protein is demonstrated in FIG. 6, which shows the immunoblot of recombinant 12 kDa protein with different grass positive sera. It was seen that Lol p, Phl p and Poa p showed higher immunoreactivity on blot followed by Imp c, Zea mays, Pennisetum and Cenchrus. This result demonstrates recombinant 12 kDa protein is an important cross-reactive allergen present in Indian and western grasses.

TABLE 1

| | | No. of | |
|---|---|---|---|
| | | patients | Specific IgE |
| S. No | Fungus | screened | value range |
| 1 | Alternaria alternata | 20 | 0.8-1.2 |
| 2 | Cladosporium herbarum | 11 | 0.7 |
| 3 | Fusarium solani | 9 | 0.8 |
| 4 | Epicoccum nigrum | 7 | 0.6-1.2 |
| 5 | Curvularia lunata | 20 | 0.9-1.6 |
| 6 | Aspergillus fumigatus | 15 | 0.8-1.0 |
| 7 | Rhizopus sp. | 10 | 0.8 |
| 8 | Mucor sp. | 8 | 0.6 |
| 9 | Pencillium citrinum | 11 | 0.7 |
| 10 | Candida albicans | 7 | 0.7 |
| 11 | Healthy controls | 20 | 0.2-0.3 |

TABLE 2

| | | | | | Specific IgE |
|---|---|---|---|---|---|
| | | | | | value to the |
| Patient | | Skin test reactivity | | | claimed 12-KDa |
| No. | Age/Sex | Fungi | Grasses | rCytc | protein |
| 1 | M/60 | −ve | Cen++±, Cyn++±, | +++ | 0.762 |
| | | | Imp++, Pen+±, | | |
| 2 | F/28 | −ve | Cen+++, Cyn++±, | +++ | 0.718 |
| | | | Pen+, Imp++ | | |
| 3 | M/27 | −ve | Cen++, Imp++, Pen+, | ++± | 0.682 |
| | | | Cyn+ | | |
| 4 | F/31 | −ve | Cen++, Imp+±, | +± | 0.318 |
| | | | Pen+++ | | |
| 5 | M/29 | −ve | Cen++±, Cyn+++, | ++± | 0.610 |
| | | | Imp++, Pen+± | | |
| 6 | F/24 | −ve | Cen+, Cyn±, Imp±, | +± | 0.412 |
| | | | Pen+ | | |
| 7 | F/23 | −ve | Cen+, Cyn±, Imp±, | +± | 0.371 |
| | | | Pen+ | | |

‘+’ = severity of the skin test reaction

### EXAMPLES

The following examples are given by way of illustration and therefore should not be construed to limit the scope of the present invention.

### Example 1

### Total RNA Isolation

One hundred mg of 4 day old CL spore mycelium mass was crushed under liquid nitrogen to obtain a fine paste. Added 1 ml of TRI zol reagent and crushed again. The paste was allowed to thaw at RT and 0.2 ml of chloroform was added to it. After gentle shaking, it was incubated for 3 m at RT and centrifuged at 12000 rpm for 15 m at 4° C. The upper aqueous layer was separated and 0.5 ml isopropanol was added and kept at −20° C. for overnight. It was centrifuged at 12000 rpm at 4° C. The pellet washed with 75% ethanol followed by centrifugation at 7500 rpm at 4° C. The pellet obtained was air dried and dissolved in 0.5% SDS. The quality of total RNA was checked on formaldehyde gel.

### Example 2

### mRNA Isolation

From purified total RNA, double oligo (dT) selection was performed to obtain poly (A) mRNA for cDNA library construction. The concentration of total RNA was adjusted to 0.55 μg/μl with DEPC treated DW and the volume was made upto 640 μl. The oligo dT washed with 1.5 ml washing buffer 1 (supplied with the kit). The salt concentration of the RNA sample was adjusted to 0.5 M by adding 64 μl of 5 M NaCl and was allowed to hybridize at RT for 10 m.

The unbound RNA was expelled and the column washed with 1.5 ml of washing buffer 1 followed by washing with buffer 2 (supplied with the kit). The poly(A) mRNA was eluted with 0.5 ml preheated (65° C.) DEPC treated DW. To the eluted 500 μl mRNA, 2 μl of 50 μg/ml glycogen, 50 μl of 7.5 M ammonium acetate and 1000 μl of chilled ethanol were added. Precipitation of RNA was carried out at −20° C. for overnight. The sample was centrifuged at 3000 rpm for 30 m at 4° C. The pellet obtained washed with 75% ethanol and centrifuged at 3000 rpm for 10 m at 4° C. The pellet was dissolved in 15 μl of DEPC treated DW.

### Example 3

### Construction of cDNA Library

The cDNA library was synthesized using Stratagene ZAP-cDNA Gigapack III Gold cloning kit. It uses a hybrid oligo dT linker primer that contains a Xho I restriction site. Messenger RNA is primed in the first strand synthesis with the linker primer. All the reagents used were provided by commercial cDNA synthesis kit. The various steps involved in the construction of the library are described below:

First strand cDNA: Messenger RNA was used as template to synthesize first strand cDNA. The reaction mixture contained 5 μg mRNA, 5 μl of 10× first strand buffer, 3 μl of 10 mM first strand methyl nucleotide mixture, 2 μl of linker primer (1.4 μg/μl) and 1 μl of RNase block (Ribonuclease inhibitor 400 U/μl) in 50 μl volume. The reaction mixture was incubated for 10 m at RT and 1.5 μl of reverse transcriptase (Moloney murine Leukemia virus reverse transcriptase, 50 U/μl) was added. The reaction was carried out at 37° C. for 1 h.

Second strand synthesis: To the first strand mix, 20 μl of 10× second strand buffer, 6 μl of second strand dNTP mixture (10 mM), 114 μl autoclaved DW, 2 μl of RNase (1.5 U/μl) and 11 μl of DNA polymerase I (9.0 U/μl) were added in a total volume of 200 μl. The reaction was carried out at 16° C. for 2.5 h and was kept on ice.

### Example 4

### Blunting the cDNA Termini

To the second strand mix, 23 μl of dNTP mix (2.5 mM) and 2 μl of cloned pfu DNA polymerase (2.5 U/μl) were added. The reaction was carried out at 72° C. for 30 m. After the completion of the incubation, 200 μl of pre saturated phenol pH-8.0: chloroform: isopropanol was added. It was mixed at RT and the upper aqueous layer was transferred into a fresh tube. To the tube, equal volume of chloroform was added and mixed. The sample was then centrifuged and the upper aqueous layer was transferred to fresh microcentrifuge tube. The DNA was precipitated by adding 20 μl of 3 M sodium acetate and 400 μl of 100% ethanol at −20° C. for overnight.

### Example 5

### Ligating the EcoRI Adaptors

The DNA pellet obtained washed with 70% ethanol and dried. To this, 1 μl of 10× ligase buffer, 1 μl of 10 mM rATP and 1 μl of T4 DNA ligase (40 U/μl) were added. The reaction was carried out at 8° C. for overnight.

### Example 6

### Phosphorylating the EcoRI Ends

After inactivating the ligase at 70° C. for 30 m, 1 μl of 10× ligase buffer, 2 μl of 10 mM rATP, 6 μl of autoclaved DW and 1 μl of T4 polynucleotide kinase (10 U/μl) were added. The reaction was carried out at 37° C. for 30 m.

### Example 7

### Digesting with Xho I

The kinase was inactivated at 70° C. for 30 m and 28 μl of Xho I buffer supplement and 3 μl of Xho I (40 U/μl) were added. The tube was incubated at 37° C. for 1.5 h. After the completion of the reaction, 5 μl of 10×STE buffer and 12 μl of 100% ethanol were added. The DNA was precipitated at −20° C. for overnight.

### Example 8

### cDNA Fractionation

The DNA pellet washed, dried and resuspended in 14 μl of 1×STE buffer followed by addition of 3.5 μl of the column loading dye was added. The drip column was packed using Sepharose CL-2B gel filtration medium and washed twice with STE buffer. After this, the cDNA sample was gently loaded without disturbing the resin. The column washed with STE buffer and cDNA sample eluates were collected. From each fraction, 5 μl of sample was aliquoted and electrophoresed on an alkaline agarose gel.

### Example 9

### Ligating cDNA into the Uni-ZAP XR Vector

The reaction mixture contained 100 ng of cDNA, 0.5 μl of 10× ligase buffer, 0.5 μl of 10 mM rATP (pH 7.5), 1 μl of UNI-ZAP XR vector (predigested, 1 μg/μl) and 0.5 μl of T4 DNA ligase (4 U/μl). The autoclaved DW was added in a total volume of 5 μl. The reaction was carried out at 12° C. for overnight.

### Example 10

### Packaging of Ligation Mixture Using Gigapack III Gold Packaging Extract

To the packaging extract, 2 μl of ligated DNA was added. After mixing it gently, the reaction mixture was incubated at 22° C. for 2 h. After the completion of incubation, 500 μl of SM buffer and 20 μl of chloroform were added. The contents were gently mixed and centrifuged at 7000 rpm at RT for 2 m. The supernatant was titrated for a suitable library dilution to be used for immunochemical screening.

### Example 11

### Plating and Titering-Blue and White Selection

Single colony of XL-1 Blue MRF′ cells was inoculated in LB containing 10 mM MgSO₄ (described in Appendix A) and 0.2% (w/v) maltose. Cells were grown at 37° C. at 220 rpm for overnight. The cells were pelleted at 4° C. at 4000 rpm for 10 m. Different library dilutions (1:10 and 1:100 v/v) were made in SM buffer. From each dilution 1 μl was taken and incubated with 200 μl of XL-1 Blue cells diluted in 10 mM MgSO₄ to 0.5 O.D₆₀₀. The mix was incubated at 37° C. for 15 m in a tube. After incubation, 2-3 ml of NZY top agar (melted and cooled to approx. 48° C., Appendix A) mixed with 5 μl of 0.5 M IPTG and X-gal were plated onto the NZY agar plates. The plates were then incubated at 37° C. for 6-8 h.

### Example 12

### Amplification of cDNA Library

XL1 Blue cells were prepared as described earlier. Primary cDNA library (250 μl containing 5×10⁴ phage particles) was incubated with 600 μl XL-1 blue cells (O.D₆₀₀ 0.5) at 37° C. for 15 m. After incubation, mixture of 6.5 ml NZY top agar and infected material was plated onto 150 mm NZY agar plates. The plates were incubated at 37° C. for 6-8 h. The plates were then overlaid with 10 ml SM buffer and stirred gently at 4° C. for overnight. The suspension was pooled in a sterile polypropylene tube. The plates were rinsed with an additional 2 ml of SM buffer and pooled. Chloroform 5% v/v was added, mixed well and incubated for 15 m at RT. The sample was centrifuged at 1000 rpm for 10 m at 4° C. and supernatant was transferred in a fresh tube. The sample was again centrifuged and supernatant was transferred in a fresh polypropylene tube. To this, chloroform was added to a final concentration of 0.3% v/v and stored at 4° C. The titer of the amplified library was checked as described earlier.

### Example 13

### Immunochemical Screening of cDNA Library

The cDNA library of C. lunata in UNI-ZAP lambda vector was screened with pre-absorbed pooled CL sensitive patient's sera. The cDNA library was plated after appropriate dilution in SM buffer for obtaining 200-300 plaques per 90 mm NZY agar plates. The cDNA library (1 μl of 1:10⁵ diluted in SM) was mixed with the host E. coli XL1-Blue MRF′ cells diluted in 10 mM MgSO₄ to OD₆₀₀=0.5 in a sterile polystyrene falcon tube. The cells were incubated at 37° C. for 15 m to allow the phage to attach to the cells. To this, 3 ml of NZY top agar (melted and cooled to 48° C.) was added and plated immediately onto NZY agar plates. The inverted plates were incubated at 42° C. (4-6 h) until the plaques just begin to form. Soaked the numbered nitrocellulose filter with 10 mM IPTG. The dried nitrocellulose filters were placed on the agar surface in contact with the plaques, taking care to avoid air bubbles under the filter. Using a syringe needle, pierced the filter and agar at asymmetric positions to facilitate paper alignment following staining. The layered plates were incubated at 37° C. for 4 h to induce expression. The filters were removed, washed twice with TBS (2 m each) and incubated in blocking buffer for 1 h at RT. After washing twice with TBS at RT (5 m each), it was incubated with serum 1:10 v/v at 37° C. for overnight. The filters were then washed and incubated in conjugate solution 1:1000 v/v in TBS at 37° C. for 3 h. The filters were washed with TBST thrice (10 m each) and color was developed. The reaction was stopped by rinsing the membranes with distilled water twice.

### Example 14

### Single-Clone Excision Protocol

The plaque showing IgE binding was cored out from the agar plate and transferred to a sterile microcentrifuge tube containing 500 μl of SM buffer and 20 μl of chloroform. Vortexed the microcentrifuge tube to release the phage particles into the SM buffer followed by incubation at 4° C. overnight (phage stock). Separate cultures of XL1 and SOLR in LB broth supplemented with 0.2% (w/v) maltose and 10 mM MgSO₄ were obtained as described earlier. On the following day, XL1 Blue and SOLR cells were spun down at 6000 rpm for 5 m at 4° C. and resuspended in 10 mM MgSO₄ at an OD₆₀₀ of 1.0. The following components were mixed in a 15 ml sterile polypropylene tube-200 μl of XL1-Blue MRF′ cells at an OD₆₀₀ of 1.0; 5 μl of phage stock and 1 μl of the ExAssist helper phage (>1×10⁶ pfu/μl). Incubated the tube at 37° C. for 15 m, added 3 ml of LB broth and incubated for 3 h at 37° C. with shaking at 220 rpm. Heated the falcon at 65° C. for 20 m and centrifuged at 10,000 rpm at 4° C. for 10 m. Decanted the phage supernatant into sterile microcentrifuge tube. To plate excised phagemids, 200 μl of SOLR cells were mixed with 2 μl of phage supernatant followed by incubation at 37° C. for 15 m. Plated 200 μl of the cell mixture on LB-ampicillin agar plates (50 μg/ml) and incubated overnight at 37° C.

### Example 15

### PCR Amplification

In general, a 50 μl of PCR reaction mixture contained,

| | | |
|---|---|---|
| 10× PCR buffer | 5.0 | μl |
| dNTP's | 4.0 | μl (0.2 mM each) |
| Forward Primer | 150 | ng |
| Reverse Primer | 150 | ng |
| Template | 10-50 | ng |
| Enzyme | 0.5 | μl (3U/μl) |

| | |
|---|---|
| DW | To make up the volume to 50 μl |

Amplification Conditions:

Initial denaturation, 94° C./5 m and added the enzyme

Denaturation, 94° C./1 m

Annealing, 55° C./2 m

Extension, 72° C./2 m

Final extension, 72° C./7′ for 25 cycles.

The size of the amplified insert was determined by agarose gel electrophoresis.

### Example 16

### Automated DNA Sequencing

Automated DNA Sequencing was performed using fluorescent dye-terminator chemistry with thermal cycle sequencing. The sequencing reaction was set up as described below: Setting up of the sequencing reaction:

| | |
|---|---|
| Components | Volume |

| | |
|---|---|
| Dye terminator Ready Reaction mix | 8 μl |
| Template DNA | Varied |
| Primer (4 pm) | Varied |
| DW | Varied |

20 μl

The thermal cycling profile (25 cycles) was as

| | |
|---|---|
| Denaturation | 96° C./10 sec |
| Annealing | 50° C./5 sec |
| Polymerization | 60° C./4 m |

Sequencing of DNA samples was performed on ABI-377, DNA Sequencer

### Example 17

### Expression and Immunological Characterization of the cDNA Encoding 12 kDa Protein

The cDNA insert subcloned into pBluescript SK (+/−) phagemid commercial kit was expressed under lacZ promoter. The phagemid was inoculated into 250 ml LB broth with 100 μg/ml of ampicillin and incubated at 37° C. with shaking (200 rpm) until the absorbance (OD₆₀₀) reached 0.2. Added IPTG at a final concentration of 1.0 mmol/L and the cultures were further grown for 5 hours at 37° C. with shaking (200 rpm). The cells were spun down at 6000 rpm for 15 min at 4° C. and suspended in 3 ml of 50 mM Tris-HCl, pH 7.5. The cells were sonicated and centrifuged at 6000 rpm for 45 min at 4° C. The supernatant was separated and was analyzed on 10% SDS-PAGE gel under reducing and denaturing conditions. After transferring the proteins onto NCM, the IgE/IgG binding activity of the fusion protein was evaluated. The patient's serum 1:10 v/v and anti CL rabbit serum 1:2000 v/v were used.

Expression of Recombinant Form of 12 kDa Protein in E.coli:

### Example 18

### PCR Reaction to Clone cDNA Encoding 12 kDa Protein

The standard PCR is typically done in 50-100 μl reaction volume and in addition to sample DNA may also contain 50 mMKCl, 10 mMTris Cl, (pH8.4), 1.5 mMMgCl₂, 250 nmoles, primers, 200 μmdNTPmix, 2.5 units of Taq DNA Polymerase.

The reaction mix used generally contained:

| | | |
|---|---|---|
| Template DNA = | 20 | ng |
| Primers = | 200 | nmoles |
| dNTPs = | 0.2 | μm |
| 10× PCR buffer = | 1× | |
| Taq DNA pol. = | 1.5U | |

The reaction mix in 50 μl volume:

| | | |
|---|---|---|
| Template = | 10 | μl |
| Primers = | 2 | μl |
| dNTPs = | 4 | μl |
| 10× PCR buffer = | 5 | μl |
| PCR grade water = | 29.5 | μl |
| Taq. pol = | 0.5 | μl |

The reaction conditions maintained were:

| | | |
|---|---|---|
| Step I-----95° C. = | 5 | min. (initial denaturation) |
| Step II-----94° C. = | 60 | sec. (denaturation of template DNA) |
| Step III----55° C. = | 60 | sec. (primer annealing to the template) |
| Step IV----72° C. = | 90 | sec. (extension by Taq pol) |
| Step V-----72° C. = | 5-7 | min. (final extension) |
| Step VI----4° C. = | 15 | min. |

The reaction (Step II-IV) was cycled 25 times.
The PCR conditions decided depend upon the particular primers used, GC content of the template DNA.

### Example 19

### Restriction Digestion

| | |
|---|---|
| Restriction enzyme buffer (10×) = | (1×) final conc. |
| DNA = | 1 μg |
| Restriction enzyme used = | 1U to completely digest |
| | 1 μg of DNA. |

For complete digestion of DNA the 50 μl reaction mix contained DNA appropriately diluted and 5 μl of assay buffer. The volume was made up by good quality autoclaved water. Finally the enzyme was added. Incubation was done at 37° C., for 3 hrs.

After first enzyme treatment, heat inactivation was done to stop its non-specific activity. The reaction mix was heated at 65° C. for 15 min. Precipitation was done by adding 0.6 volumes of ammonium acetate and 2.5 volumes of 100% ethanol. Incubate at −20° C., overnight, centrifuged and washed the pellet with 75% ethanol. The pellet was air-dried and then reaction was put up with second enzyme in similar way.

### Example 20

### Ligation

The most important thing considered during cloning is the reannealing of the cut ends, which leads to plasmid recircularization. This was prevented by phosphatase treatment that removes 5′phosphate group to suppress self-ligation. Ligase catalyzes formation of phosphodiester bonds between two nucleotides one with 3′ hydroxyl and other with 5′phosphate. This way only the foreign DNA insert can be ligated with the vector and self-ligation is minimized. The ligation mix (11 μl) contained:

5 μl of 2.2× reaction buffer and various ratios of vector and insert.0.5 μl of T4 DNA ligase was finally added. The reaction mix was incubated at 16° C., overnight.

### Example 21

### Preparation of Competent Cells

Inoculated single colony of E.coli strain to be made competent, e.g. DH5α cells into a 5 ml LB tube and grown overnight at 37° C. The next day secondary culture was done (diluted 1 ml in 100 ml of culture) Grown at 37° C. 250 rpm, 2 hrs approximately for the cells to reach OD of 0.3. OD more than 0.4 leads to decrease in competence i.e. decreases the efficiency of transformation. The culture was aliquoted into prechilled polypropylene sterile tubes and left on ice. The cells need to be kept on ice subsequently. Cells were pelleted down at 3000 rpm, 4° C. for 7 min. (higher speed affects the viability of cells). The cell pellet was suspended gently in 5-6 ml of ice-cold CaCl₂ solution (see in reagents). Cells were pelleted down at 2500 rpm, 4° C., and 5 min.

Cells were resuspended in ice cold CaCl₂ and incubated on ice for 30-40 min. Cells were pelleted down at same speed. Cell pellet was resuspended in ice cold CaCl₂. This re-suspension is final and needs to be done very well. The suspension should be kept on ice for about 1 hr. Finally the cells are aliquoted as 100 μl and stored at −70° C.

Any aliquot taken out should not be refrozen. Competence of cells is assessed by transformation with a known plasmid vector and seeing the number of colonies that appear.

No. Of transformant colonies per aliquot (μl)×105==No. of transformants per pg of DNA used for transformation.

### Example 22

### Transformation

For 100 μl of competent cells 10-20 ng of DNA usually suffices (in the volume of 10-20 μl). Competent cells with DNA were swirled gently and kept on ice for 15-20 min. This mix was then incubated at 42° C. for 2 min and immediately put on ice and kept for 5 min. This treatment is called “heat shock treatment” which actually causes DNA to enter inside the cells. The cells were revived with 300 μl of LB media and kept at 37° C., 260 rpm, 1-2 hrs. The cells were plated on LB amp plates and plates kept for overnight incubation at 37° C. Remaining part of transformation mixture can be stored at 4° C.

### Example 23

### Plasmid Isolation Alkaline Lysis (Mini Prep)

To confirm the expressed clone, plasmid isolation was carried out by alkaline lysis method. Inoculated single bacterial colony in 5 ml LB medium overnight containing appropriate antibiotic, e.g. here ampicillin (50 μg/ml). The cells were pelleted down at 600 rpm, 15 min 4° C. The cells were thoroughly mixed with 150 μl of TEG buffer by vortexing. Then cells were kept on ice for five-min. Added 300 μl of alkaline SDS was added. The solution becomes clear and slimy. Added ice-cold 200 μl potassium acetate and incubated on ice for half an hour. This step precipitates all genomic DNA and cell debris. Then centrifuged at 12000 rpm for 30 min, 4° C. Then the clear supernatant was taken out and 0.6 volumes of isopropanol was added and kept on ice for 10-15 min. Then centrifuged for 25 min at 12000 rpm, 4° C. The glassy pellet is difficult to see, thus care needs to be taken while rejecting the supernatant. The pellet was given a wash with 70% ethanol and then with 100% ethanol. The pellet was air dried and dissolved in water and analyzed on 1% agarose gel. The plasmids isolated were then checked with PCR and restriction digestion to confirm the insert of desired size.

### Example 24

### Expression and Purification of 12-kDa Recombinant Protein

The positive clone encoding 12 kDa protein was transformed into BL21 E.coli cells. The single clone was inoculated in 5 ml LB broth containing 100 μg/ml of ampicillin and incubated overnight at 37° C. with shaking (200 rpm). This culture was sub-cultured into 250 ml LB broth with 100 μg/ml of ampicillin and incubated at 37° C. with shaking (200 rpm) until the absorbance (OD₆₀₀) reached 0.2. Added IPTG at a final concentration of 1.0 mmol/L and the cultures were further grown for 5 hours at 37° C. with shaking (200 rpm). The cells were spun down at 6000 rpm for 15 min at 4° C. and suspended in 3 ml of 50 mM Tris-HCl, pH 7.5. The cells were sonicated and centrifuged at 6000 rpm for 45 min at 4° C. The sonicated lysate was loaded onto equilibrated Ni-NTA slurry and incubated for an hour for binding in equilibration buffer containing 10 mMTrsi.Cl, 100 mMsodium phosphate buffer and 500 mM NaCl pH 8.5. The non-specific bound proteins were washed off using wash buffer containing 10 mMTrsi.CI, 100 mMsodium phosphate buffer and 500 mM NaCl pH 6.2. The bound protein was eluted using wash buffer containing gradient of imidazole and finally eluted at 200 mM imidazole. The protein content was estimated by known method [9] and separated 12% SDS-PAGE gel under reducing and denaturing conditions. FIG. 1 shows the expression and purification of 12 kDa protein on SDS PAGE.

### Example 25

### SDS Page Analysis

SDSPAGE was performed by known methods [10]

| | | |
|---|---|---|
| | RESOLVING | STACKING |
| CONTENTS. | GEL(15%) | GEL(5%) |

| Acrylamide | 3.75 | ml | 600 | μl |
|---|---|---|---|---|
| Distilled water | 5.1 | ml | 2.7 | ml |
| Tris ClpH8.8 | 2.25 | ml | — | |
| TrispH6.8 | — | | 375 | μl |
| 10% SDS | 112.5 | μl | 37.5 | μl |
| Temed | 5.7 | μl | 3.75 | μl |
| 10% APS | 37.5 | μl | 13.5 | μl |

The Samples were Prepared as Follows:

30 μl eluted sample+1× sample dye (loading buffer)

Boil the samples for 10 min at 100° C. in the dry bath.

Load the samples on to the gel along with the molecular weight marker. Run the electrophoresis in 1 liter electrode buffer 1× containing 14.4 gms Glycine, 3.03 gms Tris Cl., and 1% SDS at 120V, 80 mA. The gel is stained with CBB or silver stain to visualize the protein of very little yield.

### Example 26

### Absorption Spectra of Purified Recombinant Protein

The absorption spectrum of 12 kDa protein was done to find the characteristic absorption maxima peaks of heme containing proteins and shows the peaks at 410 nm, 510 nm and 550 nm. 1 mg/ml protein was taken in a clean quartz cuvette and absorption scan carried out in the range of 210 nm-700 nm on Shimadzu UV 2100 S. the absorption maxima was recorded and the plot was scaled appropriately to fit all the peaks. FIG. 2 shows the absorption spectra of 12 kDa protein.

### Example 27

### Circular Dichroism of 12 kDa Protein

CD spectra were carried out with 1 mg/ml of each recombinant and native protein in 20 mM phosphate buffer in the far-UV range. Thermal scans in the range of 10-100° C. were also carried out to find the melting temperature (Tm) of recombinant 12 kDa protein. FIG. 3 shows the CD spectra of 12 kDa protein.

### Example 28

### Immunoblot

The protein is transferred after SDSPAGE onto nitrocellulose membrane by electrotransfer by known methods [11]. Briefly, when the run is over, the gel is transferred on to the nitrocellulose membrane sheet, in the cassette, such that the gel is on negative side and the transfer takes place from negative to positive side. The electro transfer is carried out for about 3 hrs in the transfer buffer containing 6.9 gms Glycine, 6.6 gms Tris and 250 ml methanol the volume was made upto 1 liter with distilled water) at 80 mA. After transfer is over Ponceau staining of NCP is done to see if the transfer is appropriate. Destaining of the NCP is done using PBST solution and wash with PBS. Keep the blot (NCP) in 3% BSA solution/defatted milk (made in water or PBS) used as blocking solution at 4° C., overnight or at 37° C. at 40 rpm for 1 hour. The excess blocking reagent is washed off with PBST. The blot is then incubated with primary antibody (peroxidase conjugated monoclonal antibody could be used too). The incubation could be at 4° C. for overnight or at 37° C. for 3 hours. The excess antibody is washed off with PBST. The secondary antibody is added which has to be peroxidase conjugated and kept for 1-2 hrs at 37° C. The blot is washed by PBST and kept for developing by the addition of substrate, i.e. 15 mg diaminobenzene and 15 μlH₂O₂ are added freshly to acetate buffer (0.34 g in 50 ml water+39 μl acetic acid) The blot shows brown bands upon developing if the antigen looked for is present. The allergen can also be checked similarly if the primary antibody used is serum of patient allergic to that source. FIG. 4 shows the immunoblot of 12 kDa protein using 15 patients sera.

### Example 29

### ELISA

Each well of the microtitre plate was coated with 1 μg of protein in 100 μl of coating buffer pH 9.6. The plate was incubated overnight at 4° C. After washing with 0.1 M PBS containing 0.1% Tween 20 (PBST), the free sites were blocked with 200 μl 3% bovine serum albumin or non fat dry milk for 1 h at RT. The plates were washed again and incubated with either 100 μl serum at 4° C. overnight. IgE binding was determined by allergic patients sera (1:10 v/v) and IgG binding by polyclonal mice sera/commercial antibodies (1:2000 v/v). The plate was washed again and incubated for 4 h at RT with 100 [symbol] l antihuman-IgE peroxidase (1:1000 v/v) or anti mice IgG peroxidase (1:2000 v/v). The plate washed and color was developed using substrate containing 8 mg o-phenylene diaminebenzidine and 18 μl H₂O₂ in citrate buffer at 37° C. The reaction was stopped with 50 μl 5 N H₂SO₄ after 40 m and read at 490 nm in ELISA reader (Dynatech). FIG. 5 shows the ELISA table of 12 kDa protein. For ELISA inhibition, the sera used is preincubated mix of patient's serum with different concentrations of purified recombinant or native 12 kDa protein. Rest the methodology remains the same as ELISA. FIG. 6 shows the ELISA inhibition graph. It is seen from the graph that for 50% inhibition of binding of IgE antibodies against Curvularia when incubated with native or recombinant 12 kDa protein, 7-8 ng of these proteins were sufficient. This shows that both the native and recombinant forms of 12 kDa protein are comparable immunologically and allergenically potent.

FIG. 7 further demonstrates the presence of the disclosed 12 kDa allergen in different grass extracts in immunoblot. The experiment was carried out by probing recombinant 12 kDa protein on immunoblot with different grass sensitive sera. The experiment was confirmed by probing different extracts with polyclonal antibodies raised in mice against recombinant 12 kDa protein.

Table 1 shows the specific IgE values against recombinant 12 kDa protein in different fungal positive sera. This data demonstrates the presence of detectable specific antibodies against recombinant 12 kDa protein in different fungal sensitized patient's sera.

Table 2 shows the specific IgE antibodies against the recombinant 12 kDa protein in different grass positive sera. These patients were negative to different fungi but positive to recombinant 12 kDa protein. This shows that this 12 kDa protein contributes significantly to the grass pollen allergy also. This protein can thus be useful for detection of grass and fungal allergies without using large number of grass or fungal extracts.

### REFERENCES

1. Shen H D, Lin W L, Tsai J J, Liaw S F, Han S H. Allergenic components in three different species of Penicillium: crossreactivity among major allergens. Clin. Exp. Allergy 1996; 26: 444-451.
2. Elliot M W, Taylor A J. Allergic bronchopulmonary aspergillosis. Clin. Exp. Allergy 1997; 27: 55-59
3. Valenta R, Vrtala S, Laffer S, Spitzauer S, Kraft D. Recombinant allergens. Allergy 1998; 53: 552-561
4. Chapman M D, Smith A M, Vailes L D, Arruda L K, Dhanraj V, Pomes A. Recombinant allergens for diagnosis and therapy of allergic disease. J Allergy Clin Immunol 2000; 106:409-18
5. Gupta R., Singh B P, Sridhara S., Gaur S N., Kumar R., Chaudhary V K, Arora N. Allergenic cross-reactivities of Curvularia lunata with other airborne fungal species. Allergy 2002; 57: 636-640
6. Pauli G. Evolution in the understanding of cross-reactivities of respiratory allergens: the role of recombinant allergens. Int Arch Allergy Immunol 2000; 123:183-195
7. Grendelmeier P S, Holzmann D, Himlyn M, Weichel M, Tresch S, Rückert B, Menz G,
8. Ferreira F, Blaser K, Wüthrich B, Crameri R. Native Art v 1 and recombinant Art v 1
9. are able to induce humoral and T cell-mediated in vitro and in vivo responses in mugwort allergy. J Allergy Clin Immunol 2003; 111:1328-36
9. Expression of tropomyosin from Blattella germanica as a recombinant non-fusion protein in Pichia pastoris and comparison of its IgE reactivity with its native counterpart. Jeong K Y, Lee J, Lee I Y, Hong C S Ree H, Yonga T S Protein Exp Purification 2004; 37: 273-278
10. Lowry O H, Rosebrough N J, Farr A L, Randall R J. Protein measurement with the Folin phenol reagent. J. Biol. Chem. 1951; 193:265-275.
11. Laemmli U K. Cleavage of structural proteins during the assembly of the head of bacteriophage T₄. Nature 1970; 227: 680-685
12. Towbin H, Staehelin T, Gordon J. Electrophoresis transfer of proteins from polyacrylamide gels to nitrocellulose sheets: Procedure and some applications. Proc. Natl. Acad. Sci. USA 1976; 76: 4350-4354
13. Voller A, Bidwell D, Barlette A. Microplate enzyme immunoassay for the immunodiagnosis of virus infection. In: Rose N, Feldman H (eds): Manual of Clinical Immunology. USA. American Society of Microbiology. 1976: p 506.

We claim:

1. A recombinant 12-kDa protein comprising the same or substantially the same amino acid sequence as represented by SEQ ID NO. 3.

2. A protein as claimed in claim 1, useful for the detection of respiratory allergies.

3. A protein as claimed in claim 1, wherein the protein exhibits the following characteristics:
[a] it has an iso-electric point of 9.5 as determined by iso-electric focusing,
[b] it shows UV-visible absorbance peaks at 411 nm and 511 nm,
[c] the protein has CD spectrum showing characteristic double minima in the range of 210 nm to 220 nm signifying high alpha helical content,
[d] it has melting temperature in the range of 57 to 58 degree C.,
[e] the protein is recognized by commercially available and raised specific polyclonal antibodies,
[f] it possesses allergenic activity in patients which is three to four times that in healthy controls,
[g] the protein is cross-reactive among grasses and fungi,
[h] is having comparable activity with its native form purified from fungus as confirmed by SDS-PAGE, immunoblot, ELISA, ELISA inhibition, absorbance and CD spectra.

4. A protein as claimed in claim 1, wherein the fungus used for the construction of cDNA library is selected from the group comprising of Curvularia lunata [MTCC 2030], Alternaria alternate [MTCC 1362], Epicoccum nigrum [MTCC 2129] and Fusarium solani [MTCC 1756].

5. A protein as claimed in claim 1, wherein the said protein is cross-reactive with pollen extracts of the grasses selected from the group comprising of Lolium perenne, Poa pretensis, Phleum pretense, Imperata cylindrical, Pennisetum typoides, Zea mays, Cenchrus ciliaris and the like.

6. A protein as claimed in claim 1, wherein the cDNA coding the protein sequence is represented by SEQ ID NO. 2.

7. A protein as claimed in claim 1, wherein the novel primers used for cloning the open reading frame sequence are represented by SEQ ID NOs. 4 and 5.

8. An isolated polynucleotide sequence encoding the protein as claimed in claim 1, wherein the polynucleotide sequence is a DNA.

9. A polynucleotide sequence as claimed in claim 8, wherein the sequence is represented by SEQ ID NO. 2.

10. A protein as claimed in claim 1, wherein the mRNA sequence coding the protein is represented by SEQ ID 1 (NCBI ACCESSION NO. AY034827).

11. A recombinant vector containing the DNA sequence as claimed in claim 8.

12. A diagnostic kit useful for the detection of respiratory allergies comprising the recombinant 12-KDa protein as claimed in claim 1.

13. Use of the recombinant 12-KDa protein as claimed in claim 1 for the detection of respiratory allergies.

14. Use as claimed in claim 13, wherein the protein comprises the same or substantially the same amino acid sequence as represented by SEQ ID NO. 3.

15. A method for the detection of respiratory allergies using the recombinant 12-kDa protein as claimed in claim 1, wherein the said method comprises:
[a] constructing a cDNA library of the fungus selected from Curvularia lunata [MTCC 2030], Alternaria alternate [MTCC 1362], Epicoccum nigrum [MTCC 2129] and Fusarium solani [MTCC 1756] and screening with fungus positive patient's sera;
[b] sequencing and subsequent sub-cloning of the selected cDNA fragment in an appropriate expression vector followed by expressing and purification of the cloned recombinant protein;
[c] characterizing the physiochemical and immuno-biological properties of the recombinant protein;
[d] detecting the antibodies specific to the said recombinant protein in the blood of fungal and grass allergic subjects by ELISA;
[e] validating the presence of antibodies to the said 12-kDa recombinant protein by immunoblot using hypersensitive patient's sera compared to the healthy controls.

16. A method as claimed in claim 15, wherein the fungus used for the construction of cDNA library is selected from the group consisting of Curvularia lunata [MTCC 2030], Alternaria alternate [MTCC 1362], Epicoccum nigrum [MTCC 2129] and Fusarium solani [MTCC 1756].

17. A method as claimed in claim 15, wherein the cross-reactivity of the said protein is compared with pollen extracts of the grasses selected from the group comprising of Lolium perenne, Poa pretensis, Phleum pretense, Imperata cylindrical, Pennisetum typoides, Zea mays, Cenchrus ciliaris and the like.

18. A kit useful in detection of respiratory allergy, said kit comprising primers represented by SEQ ID NO 4, SEQ ID NO 5; incorporate other components needed for utility of the kit and an instruction manual.
